(19) United States
(12) Patent Application Publication
(10) Pub. No.: US 2019/0385736 A1
(43) Pub. Date: Dec. 19, 2019

(54) OPERATION SUPPORT DEVICE AND OPERATION SUPPORT SYSTEM FOR MEDICAL INSTRUMENT

(71) Applicant: DGSHAPE Corporation, Hamamatsu-shi (JP)

(72) Inventor: Masaki HANAJIMA, Hamamatsu-shi (JP)

(21) Appl. No.: 16/443,926

(22) Filed: Jun. 18, 2019

(30) Foreign Application Priority Data
Jun. 19, 2018 (JP) 2018-115944

(51) Int. Cl. G16H 40/60; G16H 20/40; A61B 90/90

(52) U.S. Cl. CPC G16H 40/60; G16H 40/40; A61B 90/90; G16H 20/40

(57) ABSTRACT

An operation support device includes a patient information acquirer that acquires patient information in which a patient ID, a symptom, a medical practice, and an instrument ID are associated with one another from an external device, an operation storage in which an operation instruction table in which the instrument ID, the symptom or the medical practice, and an operation instruction are stored in association with one another is stored, a medical instrument reader that reads the instrument ID assigned to the medical instrument, and a processor that extracts, based on the instrument ID that has been read by the medical instrument reader, the patient information, and the operation instruction table, an operation instruction in accordance with the symptom of the patient for whom or the medical practice in which the medical instrument with the instrument ID, which has been read by the medical instrument reader, has been used.

### CROSS REFERENCE TO RELATED APPLICATIONS

This application claims the benefit of priority to Japanese Patent Application No. 2018-115944 filed on Jun. 19, 2018. The entire contents of this application are hereby incorporated herein by reference.

### BACKGROUND OF THE INVENTION

### 1. Field of the Invention

The present invention relates to an operation support device and an operation support system for a medical instrument.

### 2. Description of the Related Art

For example, as a system related to a medical instrument that is used in a medical practice, such as a surgical operation, a medical examination, or the like, a medical practice management system that manages a schedule and performance contents of a medical practice is disclosed in Japanese Patent No. 4873384. In the medical practice management system, a medical practitioner (for example, a doctor or a nurse) inputs a surgical operation schedule (which will be hereinafter referred to as “schedule information”) by selecting at least one surgical method from a plurality of surgical methods that have been set in advance. Also, in the medical practice management system, data in which information of a medical instrument that is used in a medical practice and a surgical method are associated with one another is stored. In accordance with a surgical method that has been selected by the medical practitioner, information of a medical instrument is selected.

An identification number is assigned to the medical instrument. When the medical practitioner registers performance contents of a surgical operation, the medical practitioner reads the identification number of the medical instrument that is used in the surgical operation using a reading device. Then, the identification number that has been read by the reading device is managed in association with the above described schedule information in the medical practice management system. Thus, the schedule information of the surgical operation and the performance contents, such as the medical instrument that is used in the surgical operation or the like, are associated with one another. Therefore, among data included in the performance contents, data of the above described schedule information that has been input can be used as data included in the schedule information. Thus, management of the performance contents can be facilitated.

Incidentally, depending on medical instruments, some medical instruments are washed or sterilized after being used and thus are repeatedly used for a plurality of patients. Operations of predetermined processes, such as, for example, collection, washing, assembly, sterilization, storage, or the like, are successively performed on such a medical instrument that is repeatedly used, after the medical instrument has been used. Patients include those who have infectious diseases that are easily transmitted to others and those who have diseases that are hardly transmitted to others. Therefore, there is a probability that, even using the same medical instrument, an operation that is performed in each process may differ in accordance with symptoms of a disease or the like of a patient for whom the medical instrument is used.

For example, in some cases, there is a probability that, even when the same medical instrument is used, an operation that is performed in each process differs between a medical instrument that has been used for a patient who has an infectious disease that is easily transmitted to others and a medical instrument that has been used for a patient who has a disease that is hardly transmitted to others.

### SUMMARY OF THE INVENTION

In view of the foregoing, preferred embodiments of the present invention provide operation support devices and operation support systems for medical instruments, which provide a proper operation instruction to an operator for the medical instrument that has been used.

An operation support device for a medical instrument according to a preferred embodiment of the present invention includes a patient information acquirer, an operation storage, a medical instrument reader, and a processor. The patient information acquirer is configured to acquire patient information in which a patient ID, a symptom, a medical practice, and an instrument ID assigned to the medical instrument that has been used in the medical practice are associated with one another from an external device. In the operation storage, an operation instruction table in which the instrument ID, at least one of the symptom and the medical practice, and an operation instruction are stored in association with one another is stored. The medical instrument reader is configured to read the instrument ID assigned to the medical instrument. The processor is configured or programmed to extract, based on the instrument ID that has been read by the medical instrument reader, the patient information that has been acquired by the patient information acquirer, and the operation instruction table, an operation instruction in accordance with the symptom of the patient for whom or the medical practice in which the medical instrument with the given instrument ID, which has been read by the medical instrument reader, has been used.

With an operation support device for a medical instrument according to a preferred embodiment of the present invention, in what kind of medical practice or for what kind of patient the medical instrument with the instrument ID has been used is able to be determined by acquiring the patient information from the external device. Then, the instrument ID of the medical instrument that is an operation subject is able to be acquired by reading the instrument ID of the medical instrument by the medical instrument reader. Thus, the medical practice in which or the symptom of the patient for whom the medical instrument that is an operation subject has been used is able to be acquired by comparing the instrument ID of the medical instrument that is an operation subject and the instrument ID included in the patient information to one another. Therefore, a content of a proper operation instruction in accordance with the medical practice in which or the symptom of the patient for whom the medical instrument that is an operation subject has been used is able to be extracted based on the operation instruction table. As a result, the content of the proper operation instruction for the medical instrument is able to be provided to an operator in accordance with the content of the medical practice or the symptom of the patient.

Another operation support device for a medical instrument according to another preferred embodiment of the present invention includes a patient information acquirer, a medical practice related storage, an operation storage, a medical instrument reader, and a processor. The patient information acquirer is configured to acquire patient information in which a patient ID, a symptom, a medical practice, and an instrument ID assigned to the medical instrument that has been used in the medical practice are associated with one another from an external device. In the medical practice related storage, a medical practice related table in which the medical practice and an instrument ID of the medical instrument that has been used in the medical practice are stored in association with one another is stored. In the operation storage, an operation instruction table in which the medical instrument ID, at least one of the symptom and the medical practice, and an operation instruction are stored in association with one another is stored. The medical instrument reader is configured to read the instrument ID assigned to the medical instrument. The processor is configured or programmed to extract, based on the instrument ID that has been read by the medical instrument reader, the patient information that has been acquired by the patient information acquirer, the medical practice related table, and the operation instruction table, an operation instruction in accordance with the symptom of a patient for whom or the medical practice in which the medical instrument with the given instrument ID, which has been read by the medical instrument reader, has been used.

With the another operation support device for a medical instrument according to another preferred embodiment of the present invention, the patient information is able to be acquired from the external device, and for which patient the medical instrument with the given instrument ID, which has been read by the medical instrument reader, has been used is able to be determined based on the patient information and the medical practice related table. Also, the medical practice in which or the symptom of the patient for whom the medical instrument that is an operation subject has been used is able to be acquired from the patient information. Therefore, a content of a proper operation instruction in accordance with the medical practice in which or the symptom of the patient for whom the medical instrument that is an operation subject has been used is able to be extracted based on the operation instruction table. As a result, the content of the proper operation instruction for the medical instruction is able to be provided to an operator in accordance with the content of the medical practice and the symptom of the patient.

According to preferred embodiments of the present invention, operation support devices and operation support systems for a medical instrument, each of which is able to provide a proper operation instruction to an operator for the medical instrument that has been used, are able to be provided.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a conceptual diagram illustrating an operation support system according to a first preferred embodiment of the present invention.

FIG. 2 is an explanatory diagram of a circulation cycle.

FIG. 3 is a table illustrating an example of a configuration of a patient information table.

FIG. 4 is a block diagram of an operation support device.

FIG. 5 is a table illustrating an example of a configuration of an operation instruction table.

FIG. 6 is a diagram illustrating a flow of data in procedures for instructing an operation of a sterilization process.

FIG. 7 is a block diagram of an operation support system according to a second preferred embodiment of the present invention.

FIG. 8 is a table illustrating an example of a configuration of a patient information table according to the second preferred embodiment of the present invention.

FIG. 9 is a table illustrating an example of a configuration of a medical practice related table.

FIG. 10 is a diagram illustrating a flow of data in procedures for instructing an operation of a sterilization process in the second preferred embodiment of the present invention.

### DETAILED DESCRIPTION OF THE PREFERRED EMBODIMENTS

With reference to the attached drawings, operation support systems including operation support devices for medical instruments (which will be hereinafter referred to merely as an operation support device) according to preferred embodiments of the present invention will be described below. Note that, as a matter of course, preferred embodiments described herein are not intended to be particularly limiting of the present invention. Also, members and portions that have the same function are denoted by the same reference character and redundant description will be omitted or simplified, as appropriate.

### First Preferred Embodiment

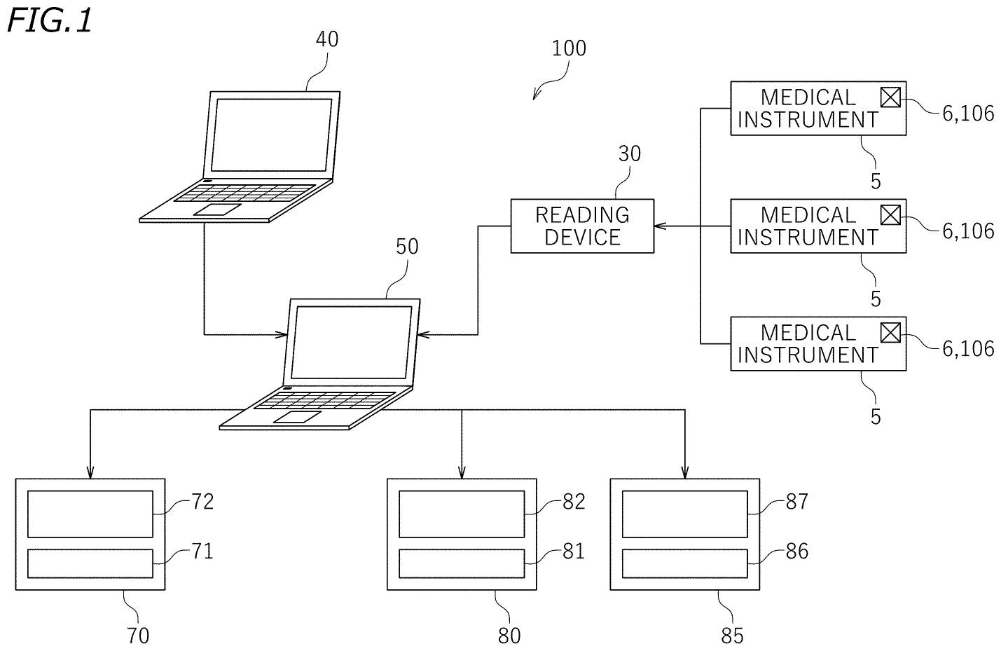

FIG. 1 is a schematic diagram illustrating an operation support system 100 according to a first preferred embodiment of the present invention. As illustrating in FIG. 1, the operation support system 100 is a system that is used to support an operation that is performed on a medical instrument 5 by an operator.

The medical instrument 5 is an instrument that is used in a medical practice that is performed in a hospital. The medical instrument 5 is, for example, a trocar, forceps, an incision device, a washing and sucking device, scissors, a scalpel (for example, a laser scalpel or an electric scalpel), an ultrasonic cutter, a scalpel holder, a cannula, tweezers, a retractor, a scale, a sonde, an elevator, a rasp, a suction tube, a rib retractor, a rib contractor, a needle holder, an injector, a metal ball, a pus tray, a cup, a pin, a mirror, a file, an opening tool, a clamp, a handpiece, an elevatorium, a chisel, a sharp spoon, a raspatory, a speculum, a suture needle, a punch, a water receiver, a needle, a penetrator, a bougie, a vent pipe, a bone impactor, a luer, a radio plier, a hammer, an angle gauge, a thermometer, a perforator, a spuit, a metal swab, an enema, a syringe, an endoscope, or the like. However, the above described examples are merely some examples of the medical instrument 5 and the medical instrument 5 is not limited to the above described examples.

The medical instrument 5 may be formed of a single part and may be formed of a plurality of parts. Examples of the medical instrument 5 formed of a plurality of parts include, for example, a trocar, forceps, an incision device, a washing and sucking device, or the like which is used in a laparoscopic surgical operation. The medical instrument 5 described above is formed of a plurality of components, is collected after a surgical operation, and is disassembled to a plurality of components. Parts that form the medical instrument 5 are referred to as components herein. Also, in some cases, such a component is further formed of a plurality of components, and in such a case, all of the components are referred to as components.

In this preferred embodiment, a medical practice is a practice related to a medical activity that is performed for a patient by a doctor. A medical practice is a practice that is performed in a medical site, that is, for example, a hospital. Examples of a medical practice include a surgical operation and a medical examination.

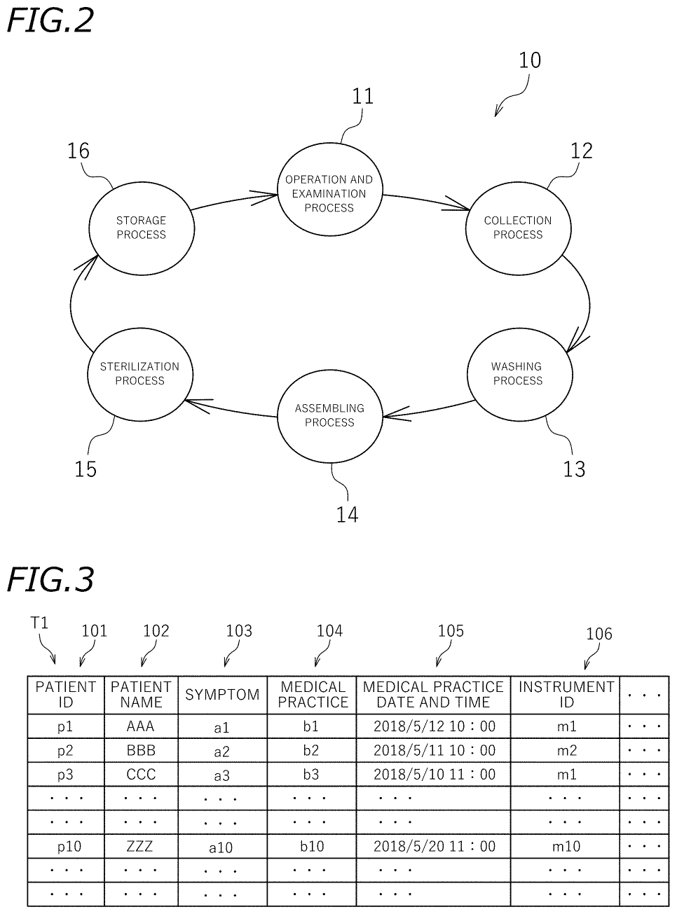

FIG. 2 is an explanatory diagram of a circulation cycle 10. As illustrated in FIG. 2, the medical instrument 5 is repeatedly usable in a predetermined circulation cycle 10. Note that the number of repetitions of the medical instrument 5 in the circulation cycle 10 is not particularly limited and differs in accordance with a type of the medical instrument 5 or a component that forms the medical instrument 5. In this preferred embodiment, the circulation cycle 10 includes an operation and examination process 11, a collection process 12, a washing process 13, an assembling process 14, a sterilization process 15, and a storage process 16.

The operation and examination process 11 is a process in which a surgical operation or a medical examination which is an example of a medical practice is performed using the medical instrument 5. In this case, in the operation and examination process 11, the medical instrument 5 that is stored in a predetermined place is taken out from the predetermined place and a surgical operation or a medical examination is performed. The collection process 12 is a process that is performed after the operation and examination process 11. The collection process 12 is a process in which, after a surgical operation or a medical examination is performed, the medical instrument 5 that has been used therein is collected. Note that, in a case in which the medical instrument 5 is formed of a plurality of components, the collection process 12 may include an operation of disassembling the medical instrument 5 to individual components. The washing process 13 is a process that is performed after the collection process 12. The washing process 13 is a process in which the collected medical instrument 5 or each of the collected individual components of the medical instrument 5 is washed. In the washing process 13, for example, using a washer 80 (see FIG. 1), washing of the medical instrument 5 is performed.

The assembling process 14 is a process that is performed after the washing process 13. The assembling process 14 is a process in which the components of the medical instrument 5 which have been washed are assembled to complete the single medical instrument 5. The assembling process 14 is a process in which the medical instrument 5 that is formed of a plurality of components is assembled. For example, for the medical instrument 5 that is formed of a single component or the medical instrument 5 that has not been disassembled in the collection process 12, the assembling process 14 may be omitted. The sterilization process 15 is a process that is performed after the assembling process 14. The sterilization process 15 is a process in which the medical instrument 5 that has been assembled is sterilized. In the sterilization process 15, for example, using a sterilizer 85 (see FIG. 1), sterilization processing of the medical instrument 5 is performed. Sterilization processing will be hereinafter also referred to merely as sterilization. The storage process 16 is a process in which the medical instrument 5 that has been sterilized is stored in the predetermined place. Note that, after the storage process 16, the operation and examination process 11 is performed.

Incidentally, in each of the processes 12 to 16 of the circulation cycle 10, there is a probability that performance contents differ for each type of the medical instrument 5. Also, even for the same medical instrument 5, there is a probability that an operation that is performed in each of the processes 12 to 16 differs in accordance with a symptom of a patient or contents of a medical practice. For example, even for the same medical instrument 5, there is a probability that a method of washing in the washing process 13 or the like differs in accordance with a symptom of a patient for whom or contents of a medical practice in which the medical instrument 5 has been used. As an example, in a case in which the medical instrument 5 is used for a patient who has a symptom indicating a high probability of infection, in the washing process 13, the medical instrument 5 may be washed by manual washing or the medical instrument 5 may be washed using a detergent having relatively high detergency. On the other hand, in a case in which the medical instrument 5 is used for a patient who has a symptom indicating a low probability of infection, in the washing process 13, even for the same medical instrument 5 described above, the medical instrument 5 may be washed by machine washing or the medical instrument 5 may be washed using a detergent having relatively low detergency. As described above, depending on the type of the medical instrument 5, in some cases, a different operation is performed for each of the processes 12 to 16. Also, even for the same medical instrument 5, in some cases, a different operation is performed for the same process of the processes 12 to 16.

In the circulation cycle 10 described above, for example, an operator is assigned each of the processes 12 to 16. The operator performs only an operation of the assigned process of the processes 12 to 16. Also, there are many types of the medical instrument 5 that is an operation subject and, conventionally, the operator has performed an operation based on his or her experiences. As described above, in some cases, performance contents have been changed in accordance with a use state of the medical instrument 5 and it has been difficult to perform a proper operation on all of the medical instruments 5.

In this preferred embodiment, the operation support system 100 supports an operation of an operator by instructing an operation in accordance with a use state of the medical instrument 5 in each of the processes 12 to 16. As will be described in detail later, in this preferred embodiment, as illustrated in FIG. 1, the operation support system 100 cooperates with an external device 40 in which patient information is stored and acquires the patient information from the external device 40. Then, the operation support system 100 instructs, based on the acquired patient information, the operator to perform optimal operation contents of the medical instrument 5 in the collection process 12, the washing process 13, the assembling process 14, the sterilization process 15, and the storage process 16.

In this preferred embodiment, as illustrated in FIG. 1, an identification label 6 is given to the medical instrument 5. The identification label 6 is used to distinguish one medical instrument 5 from another medical instrument 5. For example, an instrument ID 106 is recorded in the identification label 6. The instrument ID 106 is a character string including a number, an alphabet, or the like. The instrument ID 106 differs for each medical instrument 5. For example, the instrument ID 106 is given for each medical instrument 5. However, in a case in which the medical instrument 5 is formed of a plurality of components and is disassembled to the plurality of components in the collection process 12, the identification label 6 is provided for each of the components and the instrument ID 106 is given for each of the components. Note that there is no particular limitation on a type of the identification label 6. For example, the identification label 6 may be a one-dimensional bar code, may be a two-dimensional bar code, and may be an IC tag. In this preferred embodiment, the instrument ID 106 of the medical instrument 5 is able to be acquired by reading the identification label 6 using a reading device 30, which will be described later.

In this case, the operation support system 100 performs an operation instruction for the medical instrument 5 by reading the instrument ID 106 from the identification label 6 given to the medical instrument 5. In this preferred embodiment, as described above, the operation support system 100 supports operations for the medical instrument 5, which are performed in the collection process 12, the washing process 13, the assembling process 14, the sterilization process 15, and the storage process 16 of the circulation cycle 10. However, the operation support system 100 may be a system that supports operations for the medical instrument 5, which are performed in some of the processes 12 to 16.

In this preferred embodiment, as illustrated in FIG. 1, the operation support system 100 includes the reading device 30, the external device 40, and an operation support device 50 for the medical instrument 5 (which will be hereinafter referred to as an operation support device).

The reading device 30 reads the instrument ID 106 of the identification label 6 given to the medical instrument 5. The reading device 30 reads the instrument ID 106 recorded in the identification label 6 and transmits the instrument ID 106 that has been read by the reading device 30 to the operation support device 50. Note that there is no particular limitation on a type of the reading device 30 and the reading device 30 may be a device that can read the instrument ID 106 of the identification label 6. For example, in a case in which the identification label 6 is a two-dimensional code, the reading device 30 may be a two-dimensional code reader that can read a two-dimensional code. For example, in a case in which the identification label 6 is an IC tag, the reading device 30 may be an IC tag reader. In this preferred embodiment, the reading device 30 is a non-contact type device. However, the reading device 30 may be a contact type device. Note that the reading device 30 is provided in each of the processes 12 to 16. An installation site of the reading device 30 is, for example, in an operation space in which each of the processes 12 to 16 is performed.

The external device 40 is a separate device from each of the operation support device 50 and the reading device 30 and is electrically connected to the operation support device 50. The external device 40 is communicably connected to the operation support device 50. The external device 40 is a computer and includes a CPU, a ROM in which a program that is executed by the CPU or the like is stored, a RAM, or the like. The external device 40 may be realized by a dedicated computer and may be realized by a general-purpose computer. The external device 40 is, for example, a device that is used by a doctor.

In this preferred embodiment, an electronic medical chart of a patient is stored in the external device 40. Patient information is stored in the electronic medical chart. In other words, the patient information as the electronic medical chart is stored in the external device 40. As used herein, the “medical information” is medical information of the patient. The “medical information” is information including at least a patient ID of the patient, a symptom of the patient, medical information related to a medical practice for the patient, the instrument ID 106 given to the medical instrument 5, which has been used in the medical practice, or the like.

FIG. 3 is a diagram illustrating an example of a configuration of a patient information table T1. In this preferred embodiment, as the patient information in the electronic medical chart, the patient information table T1 (see FIG. 3) is stored in the external device 40. As used herein, a table is a table that forms a database. There are items, such as a patient ID 101, a patient name 102, a symptom 103, a medical practice 104, medical practice date and time 105, the instrument ID 106 of the medical instrument 5 that has been used in the medical practice 104, or the like, in the patient information table T1 according to this preferred embodiment. Note that the patient information table T1 may include some other item than the above described items. In this case, the symptom 103 includes a name of a disease of the patient, information of a latent virus of the patient, or the like.

Next, the operation support device 50 will be described. The operation support device 50 is a device that instructs an operator to perform an operation in accordance with a use state of the medical instrument 5 and thus supports the operation of the operator. The operation support device 50 is a computer and includes a CPU, a ROM in which a program that is executed by the CPU or the like is stored, a RAM, or the like. In this case, the operation of the operator for the medical instrument 5 is supported using a program stored in the computer. The operation support device 50 may be realized by a dedicated computer and may be realized by a general-purpose computer.

In this preferred embodiment, the operation support device 50 is electrically connected to the reading device 30 and the external device 40, and also, is communicably connected thereto. The operation support device 50 may be connected to the reading device 30 and the external device 40 via a wired communication and may be connected thereto via a wireless communication. In this case, the operation support system 100 may be a so-called client-server system, may be a cloud computing, and may be a stand-alone system.

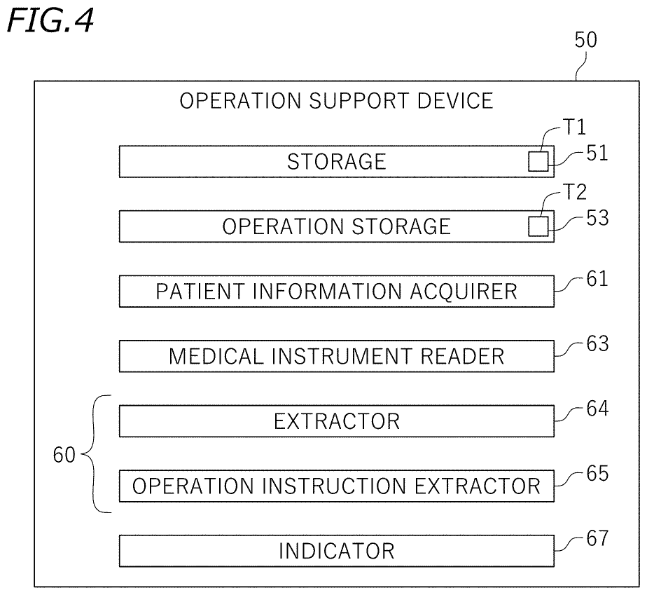

FIG. 4 is a block diagram of the operation support device 50. In this preferred embodiment, as illustrated in FIG. 4, the operation support device 50 includes a storage 51, an operation storage 53, a patient information acquirer 61, a medical instrument reader 63, a processor 60, and an indicator 67. The processor 60 includes an extractor 64 and an operation instruction extractor 65.

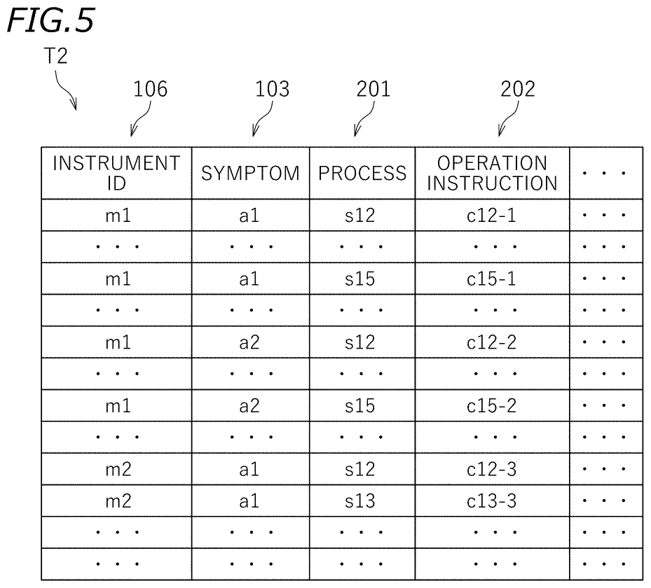

FIG. 5 is a table illustrating an example of a configuration of an operation instruction table T2. For example, the operation instruction table T2 (see FIG. 5) is stored in the operation storage 53 in advance. The operation instruction table T2 is a table in which an operation instruction 202 in accordance with the symptom 103 of a patient for whom the medical instrument 5 has been used in each of the processes 12 to 16 is indicated. As illustrated in FIG. 5, the operation instruction table T2 is a table in which the instrument ID 106 given to the medical instrument 5, the symptom 103 of the patient, a process ID 201 that indicates a process, and the operation instruction 202 for the medical instrument 5 with the given instrument ID 106 are associated with one another. In other words, there are items, such as the instrument ID 106, the symptom (for example, a disease) 103, the process ID 201, the operation instruction 202 for a process, or the like, in the operation instruction table T2. As used herein, “items associated with one another” means that items are described in the same row of the table. As for the operation instruction 202 for a process, the operation instruction 202 for the medical instrument 5 in the collection process 12 is referred to as a collection operation instruction. The operation instruction 202 for the medical instrument 5 in the washing process is referred to as a washing operation instruction. The operation instruction 202 for the medical instrument 5 in the assembling process 14 is referred to as an assembling operation instruction. The operation instruction 202 for the medical instrument 5 in the sterilization process 15 is referred to as a sterilization operation instruction. The operation instruction 202 for the medical instrument 5 in the storage process 16 is referred to as a storage operation instruction.

Note that the symptom 103 and contents of the operation instruction 202 for a process that are in the operation instruction table T2 may be managed by IDs. For example, a symptom table in which a symptom ID and a symptom are associated with one another may be stored in the storage 51 in advance and the symptom may be specified based on the symptom ID by referring to the symptom table. In this case, the item of the symptom 103 of the operation instruction table T2 is the symptom ID. In the following description, items of each table may be managed by IDs and may be managed by names. For example, each process may be managed by a process ID and may be managed by a process name.

The operation instruction table T2 of FIG. 5 may be formed of a single table and may be formed of a plurality of tables. In a case in which the operation instruction table T2 is formed of a plurality of tables, the operation instruction table T2 may include a collection operation instruction table, a washing operation instruction table, an assembling operation instruction table, a sterilization operation instruction table, and a storage operation instruction table. In this case, the collection operation instruction table is a table in which the instrument ID 106, the symptom 103, and a collection operation instruction are associated with one another. The washing operation instruction table is a table in which the instrument ID 106, the symptom 103, and a washing operation instruction are associated with one another. The assembling operation instruction table is a table in which the instrument ID 106 the symptom 103, and an assembling operation instruction are associated with one another. The sterilization operation instruction table is a table in which the instrument ID 106, the symptom 103, and a sterilization operation instruction are associated with one another. The storage operation instruction table is a table in which the instrument ID 106, the symptom 103, and a storage operation instruction are associated with one another.

In the storage 51, for example, some other table than the operation instruction table T2 (see FIG. 5) which is stored in the operation storage 53 may be stored. For example, an operation history table that is not illustrated may be stored in the storage 51. The operation history table is a table related to a history of an operation for the medical instrument 5. There are items, such as an instrument ID, a symptom, collection date and time (or a collection start date, a collection end date), washing date and time (or a washing start date, a washing end date), a washing method, assembling date and time (or an assembling start date, an assembling end date), sterilization date and time (or a sterilization start date, a sterilization end date), a sterilization method, storage date and time (or a storage start date, a storage end date), dispensing date and time (or a dispensing start date, a dispensing end date), or the like, in the operation history table. In this case, the washing method includes, for example, manual washing, single-layer automatic washing, multi-layer automatic washing, ultrasonic washing, chemical washing, or the like. The sterilization method includes, for example, autoclave (AC), plasma, gas, or the like.

The patient information acquirer 61 acquires patient information from the external device 40 in which the patient information is stored. In this preferred embodiment, the patient information acquirer 61 acquires the patient information from the external device 40 at predetermined intervals (for example, once a day or once a week). In this case, the patient information acquirer 61 transmits a patient information acquisition signal to the external device 40. The external device 40 that has acquired the patient information acquisition signal transmits the patient information to the patient information acquirer 61. In this case, the external device 40 transmits the patient information table T1 (see FIG. 3) as an example of the patient information to the patient information acquirer 61. The patient information acquirer acquires the patient information by receiving the patient information table T1. Note that, as illustrated in FIG. 4, the patient information table T1 that has been received is stored in the storage 51. The patient information acquirer 61 may be configured to acquire all of the items of the patient information table T1 of the external device 40 and may be configured to acquire only some of the items of the patient information table T1.

The medical instrument reader 63 is configured to read the instrument ID 106 given to the medical instrument 5. The medical instrument reader 63 reads the instrument ID 106 given to the medical instrument 5 by the reading device 30. For example, a reader (not illustrated) which reads the identification label 6 of the medical instrument 5 is provided in the reading device 30. The operator holds the reader of the reading device 30 over the identification label 6 of the medical instrument 5. Thus, the reading device 30 can read the instrument ID 106 recorded in the identification label 6. Note that the reading device 30 transmits, after reading the instrument ID 106, the instrument ID 106 to the medical instrument reader 63. The medical instrument reader 63 receives the instrument ID 106. Note that the instrument ID 106 that has been read by the medical instrument reader 63 is stored in the storage 51.

Note that, as described above, the reading device 30 is arranged in the space in which the operation of each of the processes 12 to 16 is performed. That is, for each of the processes 12 to 16, the dedicated reading device 30 of a corresponding one of the processes 12 to 16 is provided. For example, the process ID 201 that indicates to which one of the processes 12 to 16 the reading device is dedicated may be assigned to the reading device 30. The reading device 30 may be configured to transmit, when the instrument ID 106 of the medical instrument 5 is read by the reading device 30, the process ID 201 with the instrument ID 106 to the medical instrument reader 63. The medical instrument reader 63 receives the process ID 201 and the received process ID 201 is stored in the storage 51.

The processor 60 is configured to extract, based on the instrument ID 106 that has been read by the medical instrument reader 63, the patient information that has been acquired by the patient information acquirer 61, and the operation instruction table T2, the operation instruction 202 in accordance with the symptom 103 of the patient for whom the medical instrument 5 with the given instrument ID 106, which has been read by the medical instrument reader 63, has been used. In this case, as described above, the processor 60 includes the extractor 64 and the operation instruction extractor 65.

The extractor 64 extracts, based on the patient information table T1 that has been acquired by the patient information acquirer 61, the symptom 103 of the patient for whom the medical instrument 5 with the given instrument ID 106, which has been read by the medical instrument reader 63, has been used. Note that, in a case in which the medical instrument 5 with the given instrument ID 106, which has been read, has been used for a plurality of patients, the extractor 64 extracts, based on the medical practice time and date 105 (see FIG. 3) of the patient information table T1, the symptom 103 of one of the patients for whom the medical instrument 5 has been used most recently.

The operation instruction extractor 65 extracts, based on the symptom 103 of the patient, which has been extracted by the extractor 64, and the operation instruction table T2, the operation instruction 202 for the medical instrument 5 of the instrument ID 106 that has been read by the medical instrument reader 63. In this case, the operation instruction extractor 65 specifies the instrument ID 106 that has been read from the operation instruction table T2 by the medical instrument reader 63. Then, as illustrated in FIG. 5, the operation instruction extractor 65 extracts, based on the operation instruction table T2, the operation instruction 202 when the process ID 201 that is the process ID 201 given to the reading device 30 and is stored in the storage 51, the instrument ID 106 that has been read by the medical instrument reader 63, and the symptom 103 that has been extracted by the extractor 64 are associated with one another.

The indicator 67 instructs the operator to perform contents of the operation instruction 202 that has been extracted by the processor 60 (specifically, the operation instruction extractor 65). Note that there is no particular limitation on a method for giving an operation instruction to the operator. In this preferred embodiment, as illustrated in FIG. 1, for example, the operation support device 50 is communicably connected to a terminal 70. The terminal 70 is, for example, a computer and includes a controller 71 and a display screen 72. The terminal 70 may be a so-called tablet terminal. In this case, the indicator 67 transmits the operation instruction 202 that has been extracted by the operation instruction extractor 65 to the terminal 70. At this time, the controller 71 of the terminal 70 displays the received operation instruction 202 on the display screen 72. Note that the terminal 70 may be configured as a separate device from the operation support device 50 and may be configured to be integrated therewith.

In this preferred embodiment, as illustrated in FIG. 1, the operation support system 100 includes the washer 80 and the sterilizer 85. The operation support device 50 is communicably connected to the washer 80 and the sterilizer 85. The washer 80 is a device that is used in the washing process 13 and is a device that is used for washing the medical instrument 5. In this case, the washer 80 includes a controller 81 and a display screen 82. The sterilizer 85 is a device that is used in the sterilization process 15 and is a device that is used for sterilizing the medical instrument 5. In this case, the sterilizer 85 includes a controller 86 and a display screen 87. When the operation instruction 202 that has been extracted by the operation instruction extractor 65 is a washing operation instruction, the indicator 67 transmits the washing operation instruction to the washer 80. At this time, the controller 81 of the washer 80 displays the received washing operation instruction on the display screen 82. When the operation instruction 202 that has been extracted by the operation instruction extractor 65 is a sterilization operation instruction, the indicator 67 transmits the sterilization operation instruction to the sterilizer 85. At this time, the controller 86 of the sterilizer 85 displays the received sterilization operation instruction on the display screen 87.

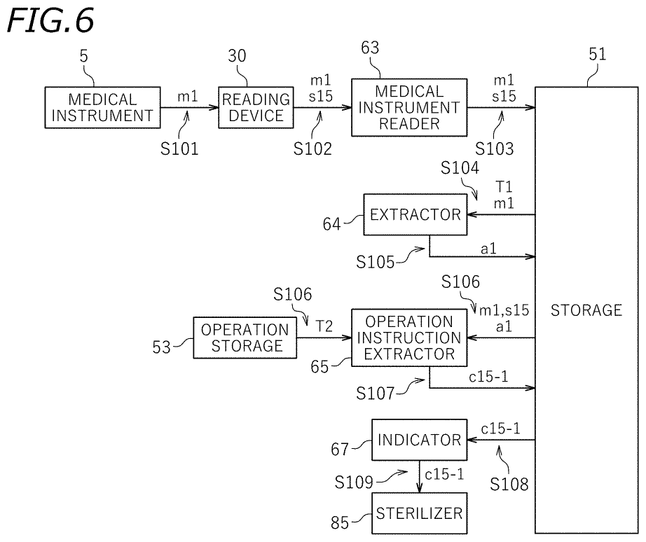

The configuration of the operation support system 100 according to this preferred embodiment has been described above. Next, procedures to support an operation for the medical instrument 5, which is performed by the operator, will be described. In this preferred embodiment, the medical instrument 5 is repeatedly used in the circulation cycle 10 (see FIG. 2). In this case, in each of the processes 12 to 16 of the circulation cycle 10, when the corresponding one of the operations of the processes 12 to 16 is started, the operator first reads the instrument ID 106 given to the medical instrument 5 using the reading device 30. After reading the instrument ID 106, the operation support device 50 indicates the optimal operation instruction 202 to the medical instrument 5 with the given instrument ID 106. The operator starts the operation along the operation instruction 202. Note that, in this preferred embodiment, in each of the processes 12 to 16, procedures from reading the instrument ID 106 given to the medical instrument 5 to extracting the operation instruction 202 are the same. Therefore, in this case, as an example, procedures to support the operation of the sterilization process 15 will be described with reference to FIG. 6. Specifically, procedures to indicate a sterilization operation instruction for the medical instrument 5 the instrument ID 106 of which is “m1” will be described.

In this preferred embodiment, before support of the operation of the sterilization process 15 is executed, the patient information acquirer 61 has acquired the patient information table T1 from the external device 40. It is assumed that the patient information table T1 that has been acquired is stored in the storage 51. In the following description, in a case of the medical instrument 5 that has not been disassembled, the medical instrument 5 means the medical instrument 5 itself. On the other hand, in a case of the medical instrument 5 that has been disassembled to a plurality of components, the medical instrument 5 means a component thereof as appropriate.

First, the operator of the sterilization process 15 holds the reading device 30 in his or her hand over the reader (not illustrated) of the reading device 30 of the identification label 6 given to the medical instrument 5 that is a subject of the sterilization. Thus, the reading device 30 reads the instrument ID 106 recorded in the identification label 6 (Step S101). In this case, “m1” of the instrument ID 106 is read from the identification label 6 and “m1” is transmitted to the medical instrument reader 63 (Step S102). Also, in Step S102, “s15” that is the process ID 201 of the sterilization process 15 is transmitted to the medical instrument reader 63. The medical instrument reader 63 stores “m1” of the instrument ID and “s15” of the process ID in the storage 51 (Step S103).

Next, the extractor 64 specifies the symptom 103 of the patient for whom the medical instrument 5 the instrument ID of which is “m1” has been used most recently from the patient information table T1 of FIG. 3 (Step S104). In this case, as illustrated in FIG. 3, in the patient information table T1, the symptom 103, the medical practice 104, the medical practice time and date 105 on which the medical practice 104 has been performed, and the instrument ID 106 of the medical instrument 5 that has been used in the medical practice 104 are associated with one another. In this case, first, the extractor 64 extracts a row in which the instrument ID 106 is “m1” from the patient information table T1 and extracts the symptom 103 of the patient in the extracted row. In this case, in a case in which the symptoms 103 of a plurality of patients is extracted, one of the symptoms 103 of the patients the medical practice time and date 105 of which is most recent is specified. In this case, in the patient information table T1 of FIG. 3, the symptoms 103 that are associated with “m1” of the instrument ID 106 are “a1” and “a3”. Of the symptoms 103, the symptom 103 the medical practice time and date 105 of which is most recent is “a1”. Therefore, in this case, as the symptom 103 the instrument ID 106 of which is “m1”, “a1” is specified. Note that “a1” of the symptom 103, which has been specified, is stored in the storage 51 (Step S105).

Next, the operation instruction extractor 65 extracts the operation instruction 202 from the operation instruction table T2 of FIG. 5 (Step S106). In this case, the operation instruction extractor 65 extracts a row in which the instrument ID 106 is “m1” and the process ID 201 is “s15” from the operation instruction table T2. The operation instruction extractor 65 extracts the operation instruction 202 in a row in which the symptom 103 of the patient is “a1” among the rows that have been extracted earlier. In this preferred embodiment, “c15-1” that is the operation instruction 202 is extracted. Note that, in “c15-1”, the proper operation instruction 202 that is the operation instruction 202 that is performed in the sterilization process 15 for the medical instrument 5 the instrument ID 106 of which is “m1” and corresponds to “a1” of the symptom 103 is indicated. In the above described manner, the operation instruction extractor 65 is able to extract contents of the operation instruction 202 in accordance with the symptom 103. Note that “c15-1” of the operation instruction 202 which has been extracted is stored in the storage 51 (Step S107).

Next, the indicator 67 extracts “c15-1” that is the operation instruction 202 stored in the storage 51 (Step S108). Then, the indicator 67 transmits “c15-1” that is the operation instruction 202 to the sterilizer 85 (Step S109). The sterilizer that has received “c15-1” of the operation instruction 202 displays the operation instruction of “c15-1” on the display screen (see FIG. 1). The operator performs sterilization processing for the medical instrument 5 the instrument ID 106 of which is “m1” in accordance with the contents related to “c15-1”.

As described above, in this preferred embodiment, in what kind of the medical practice 104 the medical instrument 5 with the given instrument ID 106 has been used is able to be determined by acquiring the patient information table T1 (see FIG. 3) from the external device 40. Then, the instrument ID 106 of the medical instrument 5 that is a subject of an operation is able to be acquired by reading the instrument ID 106 of the medical instrument 5 by the medical instrument reader 63. Therefore, the symptom 103 of the patient for whom the medical instrument 5 that is a subject of an operation has been used is able to be specified by comparing the instrument ID 106 of the medical instrument 5 that is a subject of an operation and the instrument ID 106 included in the patient information to one another. Therefore, contents of the proper operation instruction 202 that corresponds to the symptom 103 of the patient for whom the medical instrument 5 that is a subject of an operation has been used is able to be extracted based on the operation instruction table T2 (see FIG. 5). As a result, in accordance with the symptom 103 of the patient, the contents of the proper operation instruction 202 that corresponds to the medical instrument 5 is able to be provided to the operator.

In this preferred embodiment, in accordance with the symptom 103 of the patient, the operation instruction 202 for the medical instrument 5 is able to be properly instructed to the operator. Therefore, for example, even when the operator is a beginner who is not used to the operation, the operator can properly perform the operation.

In this preferred embodiment, the extractor 64 extracts the symptom 103 of the patient for whom the medical instrument 5 with the given instrument ID 106, which has been read by the medical instrument reader 63, has been used most recently, based on the medical practice time and date 105 (see FIG. 3). Thus, for example, even in a case in which the medical instrument 5 has been used for a plurality of patients, the symptom 103 of a patient for whom the medical instrument 5 has been used most recently is able to be specified. Therefore, the operation instruction 202 in accordance with the symptom 103 of the patient for whom the medical instrument 5 has been used most recently is able to be provided to the operator.

In this preferred embodiment, the medical instrument reader 63 reads the instrument ID 106 by the reading device 30 (see FIG. 1) which reads the instrument ID 106 given to the medical instrument 5. In this case, the instrument ID 106 is able to be easily read by holding the reader (not illustrated) of the reading device 30 over the identification label 6 in which the instrument ID 106 is recorded.

In this preferred embodiment, the external device 40 is a device in which an electronic medical chart is stored. The electronic medical chart includes patient information. Therefore, the symptom 103 of the patient for whom the medical instrument 5 with the given instrument ID 106, which has been read, has been used is able to be easily specified by using the patient information of the electronic medical chart. Also, it is not needed to create dedicated patient information for the operation support device 50, and therefore, an operation of newly creating patient information is able to be omitted and also management of patient information does not become complicated.

In this preferred embodiment, the symptom 103 is, for example, contents of a disease of a patient. Thus, a proper operation instruction for the medical instrument 5 is able to be performed in accordance with a type of the disease of the patient.

In this preferred embodiment, as illustrated in FIG. 5, the operation instruction table T2 includes an item of the process ID 201. To which one of the collection process 12, the washing process 13, the assembling process 14, the sterilization process 15, and the storage process 16 the operation instruction 202 is directed is able to be specified based on the process ID 201. Therefore, the proper operation instruction 202 in accordance with the processes 12 to 16 and the symptom 103 of the patient is able to be given to an operator.

In this preferred embodiment, in the washing process 13, the indicator 67 transmits the operation instruction 202 (in this case, a washing operation instruction) which has been extracted by the operation instruction extractor 65 to the washer 80. In the washer 80, the operation instruction 202 is displayed on the display screen 82 (see FIG. 1). The washer 80 is a device that is used when the operator performs washing on the medical instrument 5. Therefore, the operation support device 50 and the washer 80 are caused to cooperate with one another and the operation instruction 202 is indicated from the washer 80, and thus, the operator is able to easily use the washer 80 while looking at the contents of the operation instruction 202.

In this preferred embodiment, in the sterilization process 15, the indicator 67 transmits the operation instruction 202 (in this case, a sterilization operation instruction) which has been extracted by the operation instruction extractor 65 to the sterilizer 85. In the sterilizer 85, the operation instruction 202 is displayed on the display screen 87 (see FIG. 1). The sterilizer 85 is a device that is used when the operator performs sterilization processing on the medical instrument 5. Therefore, the operator is able to easily use the sterilizer 85 while looking at the contents of the operation instruction 202 by causing the operation support device 50 and the sterilizer 85 to cooperate with one another and indicating the operation instruction 202 from the sterilizer 85.

The operation support system 100 according to the first preferred embodiment has been described above. In the first preferred embodiment, the extractor 64 extracts the symptom 103 of the patient for whom the medical instrument 5 with the given instrument ID 106, which has been read by the medical instrument reader 63, has been used. Then, in the operation instruction table T2, the instrument ID 106, the symptom 103, the process ID 201, and the operation instruction 202 are associated with one another and the operation instruction extractor 65 extracts the operation instruction 202 that is suitable for the symptom 103 of the patient for whom the medical instrument 5 has been used, based on the operation instruction table T2. However, the operation instruction extractor 65 may be configured to extract the operation instruction 202 that is suitable for the medical practice 104 in which the medical instrument 5 has been used, based on the operation instruction table T2. In this case, in the operation instruction table T2, the symptom 103 is replaced with the medical practice 104. In this case, in the operation instruction table T2, the instrument ID 106, the medical practice 104, the process ID 201, and the operation instruction 202 are associated with one another. Also, in this case, the extractor 64 extracts the medical practice 104 in which the medical instrument 5 with the given instrument ID 106, which has been read by the medical instrument reader 63, has been used. In this case, the proper operation instruction 202 in accordance with the medical practice 104 is able to be provided to the operator.

In this case, the medical practice 104 is, for example, contents of a medical examination or a surgical operation. Thus, the proper operation instruction 202 for the medical instrument 5 is able to be performed in accordance with a type of the medial examination or the surgical operation.

### Second Preferred Embodiment

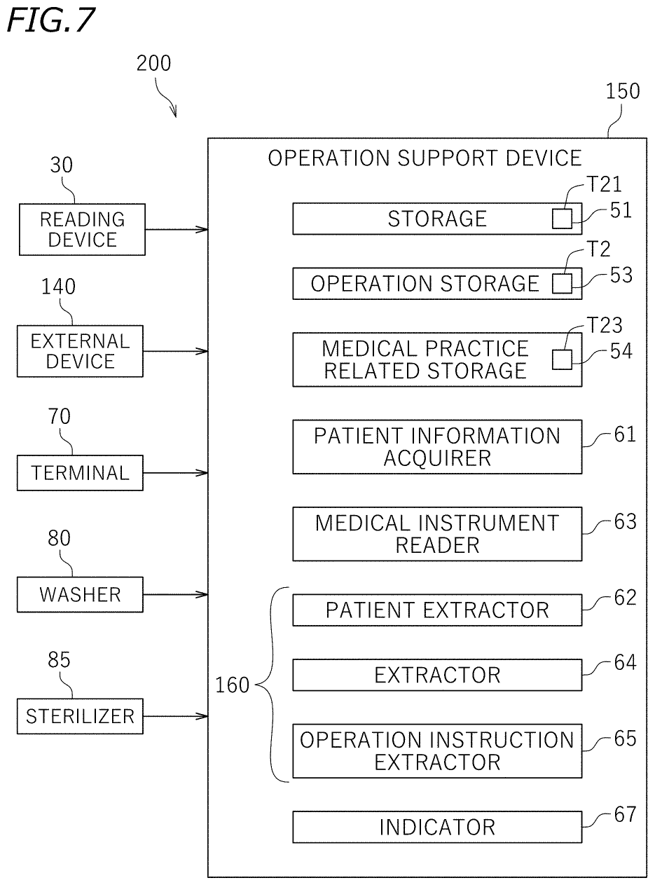

Next, an operation support system 200 including an operation support device 150 according to a second preferred embodiment of the present invention will be described. FIG. 7 is a block diagram of the operation support system 200 according to the second preferred embodiment. Similar to the first preferred embodiment, the operation support device 150 according to this preferred embodiment is communicably connected to a reading device 30, an external device 140, a terminal 70, a washer 80, and sterilizer 85. In this preferred embodiment, the reading device 30, the terminal 70, the washer 80, and the sterilizer 85 are similar to those of the first preferred embodiment, and therefore, description thereof will be omitted.

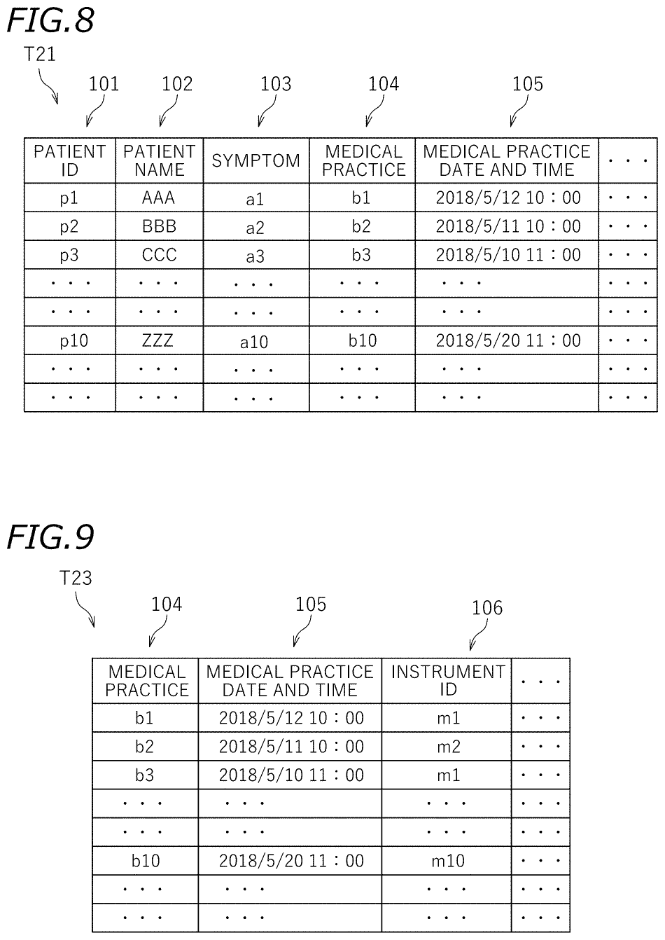

The external device 140 is a separate device from the operation support device 150. The external device 140 is a computer and is a device in which an electronic medical chart of a patient is stored. A patient information table T21 (see FIG. 8) which is an example of patient information is stored in the electronic medical chart. In this preferred embodiment, items that are included in the patient information table T21 are different from those included in the patient information table T1 (see FIG. 3) of the first preferred embodiment. In this case, there are items, such as a patient ID 101, a patient name 102, a symptom 103, a medical practice 104, a medical practice time and date 105, or the like in the patient information table T21. There is not an item of an instrument ID 106 (see FIG. 3) of a medical instrument 5 that has been used in the medical practice 104 in the patient information table T21.

In this preferred embodiment, as illustrated in FIG. 7, similar to the operation support device 50 of the first preferred embodiment, the operation support device 150 includes a storage 51, an operation storage 53, a patient information acquirer 61, a medical instrument reader 63, a processor 160, and an indicator 67. The operation support device 150 further includes a medical practice related storage 54. The processor 160 includes an extractor 64 and an operation instruction extractor 65. The processor 160 further includes a patient extractor 62.

FIG. 9 is a table illustrating an example of a configuration of a medical practice related table T23. The medical practice related table T23 (see FIG. 9) is stored in the medical practice related storage 54. In the medical practice related table T23, the medical practice 104 that is performed for a patient, the medical practice time and date 105 on which the medical practice 104 has been performed, and the instrument ID 106 of the medical instrument 5 that has been used in the medical practice 104 are associated with one another. However, some other item may be included in the medical practice related table T23. In this preferred embodiment, there are at least the items of the medical practice 104 and the instrument ID 106 in the medical practice related table T23. In this case, in the medical practice related table T23, the medical practice 104, the medical practice time and date 105, and the instrument ID 106 are associated with one another. Each item of the medical practice related table T23 is added, for example, when the medical instrument 5 is dispensed or when the medical practice 104 is performed. In this preferred embodiment, the medical practice 104 of the medical practice related table T23 and the medical practice 104 of the patient information table T21 in FIG. 8 are associated with one another in one-to-one correspondence. In this case, the medical practice 104 is, for example, provided with a unique ID, and a patient (in other words, the patient ID 101) is able to be specified based on the medical practice 104.

The processor 160 is configured or programmed to extract, based on the instrument ID 106 that has been read by the medical instrument reader 63, the patient information that has been acquired by the patient information acquirer 61, the medical practice related table T23, and the operation instruction table T2 (see FIG. 5), the operation instruction 202 in accordance with the symptom 103 of the patient for whom the medical instrument 5 with the given instrument ID 106, which has been read by the medical instrument reader 63, has been used. In this case, as described above, the processor 160 includes the patient extractor 62, the extractor 64, and the operation instruction extractor 65.

The patient extractor 62 extracts, based on the medical practice related table T23 and the patient information table T21, the patient ID 101 of the patient for whom the medical instrument 5 with the given instrument ID 106, which has been read by the medical instrument reader 63, has been used. Note that, in a case in which the medical instrument 5 with the given instrument ID 106, which has been read, has been used for a plurality of patients, the patient extractor 62 extracts the medical practice 104 in which the medical instrument 5 has been used most recently, based on the medical practice time and date 105 of the medical practice related table T23, and extracts the patient ID 101 of one of the patients for whom the medical practice 104 has been performed from the patient information table T21. Then, the extractor 64 extracts the symptom 103 of the patient of the patient ID 101 that has been extracted by the patient extractor 62, based on the patient information table T21 that has been acquired by the patient information acquirer 61.

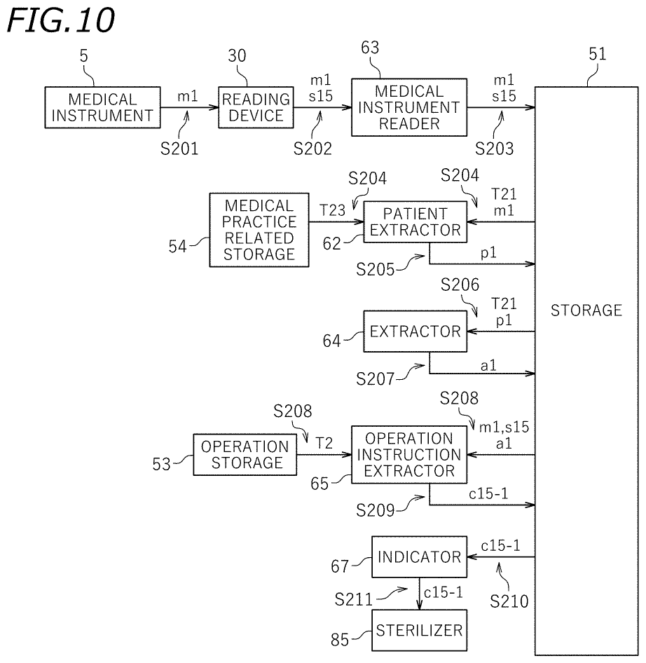

Next, in this preferred embodiment, procedures to support an operation for the medical instrument 5, which is performed by an operator, will be described. In this case, as an example, procedures to support an operation of the sterilization process 15 will be described with reference to FIG. 10. In this case, procedures to indicate a sterilization operation instruction for the medical instrument 5 the instrument ID 106 of which is “m1” will be described.

In this case, first, an operator of the sterilization process 15 reads the instrument ID 106 of the medical instrument 5 that is an operation subject using the reading device 30 (Step S201). Thus, the medical instrument reader 63 acquires, for example, “m1” as the instrument ID 106 and, at the same time, acquires “s15” that is a process ID 201 of the sterilization process 15 (Step S202). Then, the medical instrument reader 63 causes the storage 51 to store “m1” and “s15” (Step S203).

Next, the patient extractor 62 specifies the patient ID 101 of a patient for whom the medical instrument 5 with the given instrument ID 106, has been used most recently, from the medical practice related table T23 and the patient information table T21 (Step S204). First, the patient extractor 62 extracts a row in which the instrument ID 106 is “m1” from the medical practice related table T23 of FIG. 9 and extracts the medical practice 104 in the extracted row. Then, in a case in which a plurality of medical practices 104 has been extracted, the patient extractor 62 extracts the medical practice 104 the medical practice time and date 105 of which is most recent. In this case, “b1” that is the medical practice 104 is extracted from the medical practice related table T23 of FIG. 9. Next, the patient extractor 62 extracts the patient ID 101 of the patient for whom the “b1” of a medical practice 104 has been performed from the patient information table T21. In the patient information table T21 of FIG. 8, as the patient ID 101, “p1” is extracted. Note that “p1” that is the extracted patient ID 101 is stored in the storage 51 (Step S205).

Next, the extractor 64 extracts the symptom 103 of the patient of the patient ID 101 that has been extracted by the patient extractor 62, based on the patient information table T21 (Step S206). In this case, “a1” of a symptom 103 of the patient the patient ID 101 of which is “p1” is extracted from the patient information table T21 of FIG. 8. Note that “a1” that is the extracted symptom 103 is stored in the storage 51 (Step S207).

Next, the operation instruction extractor 65 extracts a row in which the instrument ID 106 is “m1” and the process ID 201 is “s15” from the operation instruction table T2 (see FIG. 5). Then, the operation instruction extractor 65 extracts the operation instruction 202 of a row in which the symptom 103 of the patient is “a1” among the rows that have been extracted earlier (Step S208). In this preferred embodiment, “c15-1” that is the operation instruction 202 is extracted and “c15-1” is stored in the storage 51 (Step S209). In a manner described above, the operation instruction extractor 65 can extract contents of the operation instruction 202. Note that Step S210 and Step S211 that are procedures performed by the indicator 67 are similar to Step S108 and Step S109 (see FIG. 6) of the first preferred embodiment, respectively, and therefore, description thereof will be omitted.

As described above, also, according to this preferred embodiment, the patient information (in this case, the patient information table T21) is able to be acquired from the external device 140, and for which patient the medical instrument 5 with the given instrument ID 106, which has been read by the medical instrument reader 63, has been used is able to be determined from the patient information table T21 and the medical practice related table T23. Also, the symptom 103 of the patient for whom the medical instrument 5 that is an operation subject has been used is able to be acquired from the patient information table T21. Therefore, contents of a proper operation instruction 202 for the symptom 103 of the patient for whom the medical instrument 5 that is an operation subject has been used is able to be extracted based on the operation instruction table T2 (see FIG. 5). As a result, the contents of the proper operation instruction 202 for the medical instrument 5 is able to be provided to the operator in accordance with the contents of the symptom 103 of the patient.

In this preferred embodiment, the patient extractor 62 extracts, based on the medical practice time and date 105 of the medical practice related table T23, the medical practice 104 in which the medical instrument 5 with the given instrument ID 106, which has been read by the medical instrument reader 63, has been used most recently and extracts, based on the patient information table T21, the patient ID 101 of the patient for whom the medical practice 104 has been performed. Thus, for example, even in a case in which the medical instrument 5 has been used for a plurality of patients, the patient ID 101 of the patient for whom the medical instrument 5 has been used most recently is able to be specified. Accordingly, the operation instruction 202 in accordance with the symptom 103 of the patient for whom the medical instrument 5 has been used most recently is able to be provided to the operator, based on the specified patient ID 101.

The operation support system 200 according to the second preferred embodiment has been described above. In the second preferred embodiment, the extractor 64 extracts a symptom 103 of a patient for whom the medical instrument 5 with the given instrument ID 106, which has been read by the medical instrument reader 63, has been used. In the operation instruction table T2, the instrument ID 106, the symptom 103, the process ID 201, and the operation instruction 202 are associated with one another and the operation instruction extractor 65 extracts an operation instruction that is suitable for the symptom 103 of the patient for whom the medical instrument 5 has been used, based on the operation instruction table T2. However, the operation instruction extractor 65 may be configured to extract, based on the operation instruction table T2, the operation instruction 202 that is suitable for the medical practice 104 in which the medical instrument 5 has been used. In this case, in the operation instruction table T2, the symptom 103 is replaced with the medical practice 104. In this case, in the operation instruction table T2, the instrument ID 106, the medical practice 104, the process ID 201, and the operation instruction 202 are associated with one another. Also, in this case, the extractor 64 extracts the medical practice 104 in which the medical instrument 5 with the given instrument ID 106, which has been read by the medical instrument reader 63, has been used.

In each of the above described preferred embodiments, the corresponding one of the external devices 40 and 140 is a device in which an electronic medical chart is stored. However, each of the external devices 40 and 140 is not limited to a device in which an electronic medical chart is stored, as long as each of the devices 40 and 140 is a device in which the patient information is stored. For example, each of the external devices 40 and 140 may be a device in which so-called surgical information is stored. A device in which the surgical information is stored is a so-called surgical system. Patient information is stored in the surgical information.

Note that each of the storage 51, the operation storage 53, the patient information acquirer 61, the medical instrument reader 63, the processor 60 (specifically, the extractor 64 and the operation instruction extractor 65), and the indicator 67 each of which is a component of the operation support device 50 of the first preferred embodiment, the storage 51, the operation storage 53, the medical practice related storage 54, the patient information acquirer 61, the medical instrument reader 63, the processor 160 (specifically, the patient extractor 62, the extractor 64, and the operation instruction extractor 65), and the indicator 67 each of which is a component of the operation support device 150 of the second preferred embodiment may be configured by a software. That is, a computer program may be read by a computer, and thus, each of the above described components may be realized by the computer. Preferred embodiments of the present invention include computer programs that cause computers to function as each of the above described components. Also, preferred embodiments of the present invention include computer-readable recording media in each of which the computer program(s) is recorded. Also, each of the above described components may be a processor that is configured or programmed to execute a computer program that is stored in each of the operation support devices 50 and 150. In this case, each of the components may be realized by one processor and may be realized by a plurality of processors. Also, preferred embodiments of the present invention include circuits in each of which a similar function to a program that is executed by each component is realized.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. An operation support device for a medical instrument,
the operation support device comprising:
a patient information acquirer to acquire from an external device patient information in which a patient ID, a symptom, a medical practice, and an instrument ID assigned to the medical instrument that has been used in the medical practice are associated with one another;
an operation storage in which an operation instruction table in which the instrument ID, at least one of the symptom and the medical practice, and an operation instruction are stored in association with one another, is stored;
a medical instrument reader to read the instrument ID assigned to the medical instrument; and
a processor to extract, based on the instrument ID that has been read by the medical instrument reader, the patient information that has been acquired by the patient information acquirer, and the operation instruction table, an operation instruction in accordance with the symptom of the patient for whom or the medical practice in which the medical instrument with the instrument ID, which has been read by the medical instrument reader, has been used.

2. The operation support device for a medical instrument according to claim 1, wherein the processor includes:
an extractor to extract, based on the patient information that has been acquired by the patient information acquirer, the symptom of the patient for which or the medical practice in which the medical instrument with the instrument ID, which has been read by the medical instrument reader, has been used; and
an operation instruction extractor to extract, based on the symptom of the patient or the medical practice which has been extracted by the extractor and the operation instruction table, the operation instruction for the medical instrument with the instrument ID, which has been read by the medical instrument reader.

3. The operation support device for a medical instrument according to claim 2, wherein
a medical practice date and time which is a date and a time on which the medical practice has been performed is associated with the patient information; and
the extractor extracts, based on the medical practice date and time, the symptom of the patient for which or the medical practice in which the medical instrument with the instrument ID, which has been read by the medical instrument reader, has been used most recently.

4. An operation support device for a medical instrument, the operation support device comprising:
a patient information acquirer to acquire from an external device patient information in which a patient ID, a symptom, a medical practice, and an instrument ID assigned to the medical instrument that has been used in the medical practice are associated with one another;
a medical practice related storage in which a medical practice related table in which the medical practice and an instrument ID of the medical instrument that has been used in the medical practice are stored in association with one another is stored;
an operation storage in which an operation instruction table in which the medical instrument ID, at least one of the symptom and the medical practice, and an operation instruction are stored in association with one another, is stored;
a medical instrument reader to read the instrument ID assigned to the medical instrument; and
a processor to extract, based on the instrument ID that has been read by the medical instrument reader, the patient information that has been acquired by the patient information acquirer, the medical practice related table, and the operation instruction table, an operation instruction in accordance with the symptom of a patient for whom or the medical practice in which the medical instrument with the instrument ID, which has been read by the medical instrument reader, has been used.

5. The operation support device for a medical instrument according to claim 4, wherein the processor includes:
a patient extractor to extract, based on the patient information that has been acquired by the patient information acquirer and the medical practice related table, a patient ID of the patient for whom the medical instrument with the instrument ID, which has been read by the medical instrument reader, has been used;
an extractor to extract, based on the patient information that has been acquired by the patient information acquirer, the symptom of the patient or the medical practice which corresponds to the patient ID that has been extracted by the patient extractor; and
an operation instruction extractor to extract, based on the symptom of the patient or the medical practice which has been extracted by the extractor and the operation instruction table, the operation instruction for the medical instrument with the instrument ID, which has been read by the medical instrument reader.

6. The operation support device for a medical instrument according to claim 5, wherein
a medical practice date and time which is a date and time on which the medical practice has been performed is associated with the medical practice related table; and
the patient extractor extracts, based on the medical practice date and time of the medical practice related table, the medical practice in which the medical instrument with the instrument ID, which has been read by the medical instrument reader, has been used most recently and extracts, based on the patient information that has been acquired by the patient information acquirer, the patient ID of the patient for whom the medical practice has been performed.

7. The operation support device for a medical instrument according to claim 1, wherein the medical instrument reader reads the medical ID assigned to the medial instrument by a reader that reads the instrument ID assigned to the medial instrument.

8. The operation support device for a medical instrument according to claim 1, wherein the external device is a device in which an electronic medical chart is stored.

9. The operation support device for a medical instrument according to claim 1, wherein the symptom is a content of a disease of the patient.

10. The operation support device for a medical instrument according to claim 1, wherein the medical practice is a content of a medical examination or a surgical operation.

11. The operation support device for a medical instrument according to claim 1, wherein
in the operation instruction table, the instrument ID, the at least one of the symptom and the medical practice, a collection process in which collection is performed, and a collection operation instruction that is an operation instruction for the medical instrument in the collection process are associated with one another; and
the processor extracts, based on the operation instruction table, the collection operation instruction for the medial instrument with the instrument ID, which has been read by the medial instrument reader.

12. The operation support device for a medical instrument according to claim 1, wherein
in the operation instruction table, the instrument ID, the at least one of the symptom and the medical practice, a washing process in which washing is performed, and a washing operation instruction that is an operation instruction for the medical instrument in the washing process are associated with one another; and
the processor extracts, based on the operation instruction table, the washing operation instruction for the medial instrument with the instrument ID, which has been read by the medial instrument reader.

13. The operation support device for a medical instrument according to claim 12, further comprising:
an indicator to indicate an operation by transmitting the washing operation instruction that has been extracted by the processor to a washer.

14. The operation support device for a medical instrument according to claim 1, wherein
in the operation instruction table, the instrument ID, the at least one of the symptom and the medical practice, a sterilization process in which sterilization is performed, and a sterilization operation instruction that is an operation instruction for the medical instrument in the sterilization process are associated with one another; and
the processor extracts, based on the operation instruction table, the sterilization operation instruction for the medical instrument with the instrument ID that has been read by the medical instrument reader.

15. The operation support device for a medical instrument according to claim 14, further comprising:
an indicator to instruct an operation by transmitting the sterilization operation instruction that has been extracted by the processor to a sterilizer.

16. The operation support device for a medical instrument according to claim 1, wherein
in the operation instruction table, the instrument ID, the at least one of the symptom and the medial practice, an assembling process in which assembling is performed, and an assembling operation instruction that is an operation instruction for the medical instrument in the assembling process are associated with one another; and
the processor extracts, based on the operation instruction table, the assembling operation instruction for the medical instrument with the instrument ID that has been read by the medical instrument reader.

17. The operation support device for a medical instrument according to claim 1, wherein
in the operation instruction table, the instrument ID, the at least one of the symptom and the medical practice, a storage process in which storage is performed, and a storage operation instruction that is an operation instruction for the medical instrument in the storage process are associated with one another; and
the processor extracts, based on the operation instruction table, the storage operation instruction for the medical instrument with the instrument ID that has been read by the medical instrument reader.

18. An operation support system for a medical instrument, the operation support system comprising:
the operation support device for a medical instrument according to claim 1; and
a reader that reads the instrument ID.
